(19) United States
(12) Patent Application Publication
(10) Pub. No.: US 2021/0170145 A1
(43) Pub. Date: Jun. 10, 2021

(54) MOUNTING DEVICE FOR ADAPTING A FOLEY URINARY CATHETER AS A PERCUTANEOUS ENDOSCOPIC GASTROSTOMY (PEG) CATHETER FOR ENTERAL FEEDING

(71) Applicant: PONTIFICIA UNIVERSIDAD CATOLICA DE CHILE, Santiago (CL)

(72) Inventors: Mariela Isabel CAMPOS STEMPFLE, Santiago (CL); Valentina Constanza MARQUEZ VILLABLANCA, Santiago (CL); Fernanda Sofía ROMAGNOLI TORRES, Santiago (CL); Iván Alberto CARO PINTO, Santiago (CL); Juan Carlos PATILLO SILVA, Santiago (CL); José Fernando Enrique VULETIN SOLIS, Santiago (CL); Alejandro Luis ZAVALA BUSQUETS, Santiago (CL)

(73) Assignee: PONTIFICIA UNIVERSIDAD CATOLICA DE CHILE, Santiago (CL)

(21) Appl. No.: 17/263,064

(22) PCT Filed: Jul. 3, 2019
(86) PCT No.: PCT/CL2019/050054

§ 371 Date: Jan. 25, 2021

(30) Foreign Application Priority Data
Jul. 24, 2018 (CL) 1999-2018

(51) Int. Cl. A61M 25/02

(52) U.S. Cl. CPC A61M 25/02; A61M 2025/024; A61M 2202/0482; A61M 2210/1021; A61M 2025/0233

(57) ABSTRACT

The invention relates to a mounting device for adapting a Foley urinary catheter as a percutaneous endoscopic gastrotomy (PEG) catheter for enteral feeding, which comprises a clamp-type radial retention means that holds the catheter to prevent it from migrating into the patient's stomach cavity, said clamp being secured in its position by a locking button for coupling laterally to the catheter and by a perimeter closure ring that is pressed onto the outside of the locking button.

The invention subject of the present Patent of Invention Application is applicable in the field of medicine and refers to a mounting device for adapting a Foley urinary catheter (A) as a percutaneous endoscopic gastrostomy (PEG) catheter for enteral feeding.

### DESCRIPTION OF PRIOR ART

Gastrostomy is a surgical procedure that creates a temporary or permanent direct feeding pathway between the stomach and the skin of a patient's abdominal wall. Specifically, it consists of the opening of a hole in the anterior wall of the abdomen to introduce a feeding catheter into the stomach.

It is a safe method that is considered of choice in situations of prolonged enteral nutrition and is used mainly to allow direct feeding to patients who cannot accept food by mouth; for example, gastrostomy is often required for patients who have dysphagia (difficulty swallowing food) due to a mechanical or neurological problem and who are presumed to have an adequate life expectancy.

Gastrostomies are generally performed by endoscopic placement of a feeding tube in which a wire passes into the stomach through the abdominal wall and the wire is retrieved with an orally inserted endoscope.

Known catheters have at their distal end an internal retention device with which the catheter flexible tube rests on the inner wall of the stomach; the flexible tube of the catheter is sized to extend beyond the abdominal wall. At its proximal end, the flexible tube has a connection piece to be able to connect the transfer system to provide the nutritive solution.

However, many times these procedures and devices used turn out to be very expensive for an important group of patients who do not have the economic resources to pay for it, as well as being an expensive device for public institutions that provide medical services of this type.

For this reason, one of the basic solutions that has been sought to apply is related to the use of a urinary catheter, normally known as a Foley catheter in gastrostomy applications, mainly because the Foley catheter is very low cost, so that has sought to solve the problem of installing it through an abdominal wall.

The main problem that arises has to do with the fact that the Foley catheter is designed for urinary use being installed through the urinary ducts until it reaches the patient's bladder to extract the accumulated urine. So it is a long and very flexible tube, usually made of latex or silicone, which at its proximal end has one, two or three branches through which air or serum is introduced to inflate a retention mechanism arranged at the opposite distal end or the urine collection bag is connected, and in the case where there are three branches, one of them is used to introduce washing fluid.

At the opposite end where the inflatable balloon is, there is also an eyelet through which the urine is extracted from the bladder; meanwhile, outside the patient, the section of the catheter that comes out is attached to the body with adhesive tapes and is connected to the collection bag.

Normally, in the outer area of the patient's abdomen, the catheter has external holding or retention means that seek to stabilize the catheter on the abdominal wall, preventing axial displacements from occurring, since these displacements not only carry the possibility of contracting infections, but also produce unexpected and unwanted leaks for the patient.

Since the Foley catheter is to be introduced through the urinary tract, it does not have any external means of support that fixes its position and prevents a retraction movement into or out of the body; therefore, when it comes to using this type of catheter for gastrostomy applications, the problem arises of how to fix the catheter outside the opening made in the patient's skin to connect the catheter with the inside of the stomach and related organs.

One of the solutions that can be found in practice is the placement and fixation of an external attachment other than the catheter that allows it to be immobilized on the skin, sometimes finding solutions as precarious as a piece of another larger catheter with a longitudinal cut where the Foley catheter is made to pass and when it is transversal to it, it is fixed with adhesives to the outside of the skin. This solution is very basic and prevents a secure fixation of the catheter, so it is of short-term use and must be reviewed regularly preventing the patient from having a relatively normal life within which means the use of a catheter to feed.

In the state of the art, it is possible to find Foley-type catheters to which a fixed disk has been incorporated that serves to slow down the migration of the catheter into the patient, as described in the US Patent document U.S. Pat. No. 4,666,433, in it a catheter similar to a Foley catheter can be seen, which has an external fixed disc that rests on the patient's skin but this solution is not very effective because the disc is fixed with pre-established measurements on the distance between the inner retainer and the outer clamping disc, limiting the possibilities of coupling to different types of patients; specially the problem increases because the distance between the clamping pieces must be adjusted to the thickness of the abdominal walls, including adipose and muscular tissue but these layers are usually reduced by the same effect of a feeding system that is not natural, so it is common to see that the initial adjustment between the inner retainer and the outer disk is insufficient after a short time, appearing clear leaks and sometimes even the complete detachment of the catheter.

To overcome the previous problem, solutions have been seen that allow more than one fixing position, with certain steps between one length and another allowing the disc to be moved in each of these positions, as described in the North American Patent Application US2016/0302999; however, as the length sections of the catheter are preset it also limits the use in intermediate positions and would make it difficult to use the same catheter to adjust it to the patient's requirement.

As it is possible to see, the invention described above cannot solve the fixation of the catheter in a really desired position because it may happen that an intermediate step is required which is not foreseen at the predetermined points, so that each time the catheter remains loose implies having to hold the disc and pull the catheter out so that the next stop is fixed, a fact that implies having to control how much force to apply on the catheter traction, that it is not too much and the disc is fixed in one position too tight that compresses the abdominal walls excessively or causes the retention balloon to escape from the cavity.

It has also been a drawback in active and mobile patients the fact that the flexible tube of the catheter protrudes to a relatively large extent from the abdominal wall, so that solutions have also been developed that seek to fold the catheter outside the abdominal wall, while cooperating with axial fixation and preventing the catheter from migrating into the patient; one of these solutions is seen in the North American Patent U.S. Pat. No. 5,267,969 where a fixation device is described in which the catheter is inserted and folded at a right angle, where this device has a lower wall that is in contact with the patient's skin preventing the catheter from easily moving, at the same time lowering the outer projection profile forcing the catheter o remain attached to the body.

The problem with this prior art solution is that it is very difficult to assemble, since it involves folding the catheter before inserting it having to set in advance an adequate distance between the device and the internal retention balloon, which is difficult to calculate since it is a thickness that varies according to the different layers of the abdomen, thus, once installed, if it is loose it makes its adjustment difficult because the catheter is already folded and must move in the same folded position.

This and other devices have proven to be useful products in that they are low profile, that is, essentially flush with the skin thus avoiding the problems associated with extended length feeding tubes and also because they include one-way valves that prevent the reflux of gastric contents (reflux). However, despite the usefulness of such products, the length of the device is likely to vary from patient to patient; therefore, hospitals must maintain an inventory of different sizes as the available devices are not easily adjustable in size.

Therefore, it is deduced the need for a mounting device that allows the adaptation of a low cost Foley type catheter to be applied as a gastrostomy catheter, which allows the distance between the internal retention balloon and the surface of the patient's skin, as well as that this device is capable of maintaining a low profile of external projection of the catheter, keeping it as close to the patient as possible, so that the patient can develop relatively normally without this catheter protruding noticeable and is hidden under clothing without being annoying.

### DESCRIPTION OF THE INVENTION

The present invention relates to a mounting device for adapting a urinary catheter, Foley type, as a percutaneous endoscopic gastrostomy (PEG) catheter for enteral feeding.

One of the main objects of the present invention is to provide a Foley type catheter mounting device that allows the position of the catheter to be fixed outside the patient, preventing it from migrating into the abdominal cavity.

Another main object of the invention is to provide a Foley type catheter mounting device that allows the distance between the internal retention balloon and the outer wall of the patient's abdomen to be universally adjusted including millimeter adjustments.

Still another of the main objects of the invention is to provide a Foley type catheter mounting device that allows its adjustment on repeated occasions as it is released due to a natural change in the patient's conditions, such as thinning of the abdominal walls or loss of fluid in the internal retention balloon.

Another object of the invention is to provide a Foley type catheter mounting device that allows the catheter to be retained by exerting radial compression on it without risks of breakage or obstruction of the passage of the nutrient for enteral feeding.

Another object of the invention is to provide a Foley type catheter mounting device that allows blocking the displacement in opposite directions of the parts that generate radial retention of the catheter, ensuring the compression and fixation of the probe so that it does not move axially and loses the adjusted position relative to the skin of the patient's abdominal area.

Another object of the invention is to provide a Foley type catheter mounting device that allows the parts to be secured in a closed position, but at the same time, allows their reversal and opening to re-adjust the device in case it is necessary.

Therefore, the present invention provides a mounting device for adaptation of a urinary catheter, Foley type, as a percutaneous endoscopic gastrostomy (PEG) catheter for enteral feeding, which comprises a radial retention means of the clamp type of the catheter to prevent it from migrating into the patient's stomach cavity, which is formed by clamp pieces that leave a central axial perforation where the catheter is compressed and that have interior walls having an angle that adjusts and prevents vertical displacement of clamp pieces to each other, which in turn is secured by a locking button that engages laterally to the catheter and holds the clamp pieces together, this locking button being secured in position by a perimeter locking ring (30) that is pressed outside the locking button (20) and secures it against the clamp pieces.

The clamp has the aforementioned central axial perforation in whose walls it has internal radial compression grooves that stabilize the catheter to prevent its axial displacement and that it migrates into the abdominal cavity; the central axial hole comprises an internal diameter that varies between 5 mm. and 6 mm., being preferably 4 mm.

The mentioned grooves protrude radially from the wall of the central axial hole generating a distance between them that is less than the outer diameter of a catheter, so that once the clamp surrounds the catheter and houses it in its axial hole, said grooves radially compress the catheter trapping it and preventing it from moving.

These grooves can have a different cross section, such as a triangular or trapezoidal section, but in a preferred embodiment it is semi-circular in section.

The clamp comprises means for retaining the locking button and means for receiving the perimeter closure ring, where the means for retaining the locking button are given by an outer wall of the clamp which is rounded in its rear area and with a converging negative tilt from top to bottom, around which the locking button adjusts and engages with a lateral displacement. In turn, the means for receiving the perimeter closure ring comprise a horizontal channel located on the upper edge of the front part of said clamp which has a convex curved path.

This clamp comprises an upper face normally flat, and a lower face also flat, from whose edges a circular perimeter spigot projects forming part of the lower base of the device through which it makes contact with the patient's skin just around the opening where the catheter passes into the gastric cavity.

Said clamp is divided into two pieces, a major base piece laterally attachable to a minor base piece, where the major base piece has an inner wall facing an inner wall of the minor base piece and where the upper face of it has a surface larger than the surface of the upper face of the minor base piece.

Said inner wall of the major base piece is folded inward at a concave angle, which is complementary to the inner wall of the minor base piece, which is folded outward at a convex angle, so that when both base pieces are coupled with one another surrounding the catheter, it remains housed in the central axial hole with the grooves, while the interior walls folded at an angle are coupled and due to the angle they present, they remain relatively immobilized with each other preventing an easy vertical movement between them; thus, this adjustment of the walls at an angle to each other ensures that when the clamp is coupled both pieces acquire the same relative height concerning to the patient's skin.

Both base pieces of the clamp comprise outer radial flaps projected from the perimeter projection, normally of such a thickness that they acquire semi-flexible characteristics and when projecting from the base of the clamp they are those that cooperate in the adaptation of the mounting device on the skin of the patient, whereby the flaps comprise an upper face and a lower opposite face that is in contact with the skin. Each of these flaps has an elongated end shape and rounded lower edges to ensure harmless contact with the skin and that they do not bury, in the same way, its elongated shape allows a fastening to the skin with the least possible surface in contact reducing the chances of irritation either by the adhesive means that are placed or by some fluid that has leaked from the opening in the abdomen.

To solve the problem of the amount of catheter that remains outside the device and that generates a large volume on the outside, at least one of the flaps comprises at a distal end a hook with a through hole to hold the catheter in the folded state, which not only holds the catheter to keep it low profile close to the user but also acts as a means of releasing tension from the catheter, such that if the probe is accidentally pulled, the pull is not transmitted directly to the opening made in the abdominal wall for the passage of the catheter but rather that traction is supported by the hook preventing an accidental exit or irritation of the wound through which the catheter enters.

The aforementioned locking button that forms part of this mounting device comprises a substantially cylindrical body with an outer mantle preferably smooth and straight with a flat lower face and an upper face that is also preferably flat. In this body of the locking button three axial portions are distinguished, formed by an upper portion, an intermediate portion and a lower portion, where only the upper portion comprises an axial central hole from which a radial straight recess extends to the outer mantle of the body, where this perforation has an internal diameter that is coincident with the diameter of the central axial perforation of the clamp and it is through it that the catheter passes in the assembly process.

On the other hand, the intermediate portion and the lower portion of the locking button comprise a substantially vertical radial recess originating from a frontal area of the mantle but this radial recess has its inner wall with a positive inclination diverging from the bottom up, which is coincident in shape, size and inclination with the shape, size and inclination of the inclined outer wall of the clamp, allowing the locking button to engage laterally in the clamp and to be retained in it by the effect of the inclined walls, preventing its vertical axial displacement so that once the locking button is placed with a lateral displacement with respect to the catheter and the clamp said inclined walls lock each other by the negative inclination and prevent said button from being displaced axially ensuring locking of the two base pieces of the clamp.

In said locking button, the intermediate portion comprises two horizontal recesses opposed to each other where each of them extends around the perimeter for approximately a quarter of a circumference from the entrance of the rearward radial recess that has the sloping walls and its position in the height of the locking button coincides with the height position of the horizontal curved path front channel arranged in the front upper edge of said clamp, so that when the locking button is laterally coupled to the clamp, the perimeter horizontal recesses of the button are aligned with said front horizontal channel of the clamp forming a single continuous channel whose purpose is to receive the coupling of said perimeter closure ring.

This ring has the shape of an open hoop with an internal diameter that coincides with the diameter of the locking button at the bottom of the horizontal recesses that appear in the mantle of the intermediate portion; on the other hand, in this open ring there are two curved arms and a central frontal projection that has a semicircular outer notch through which the catheter passes.

This ring has a total perimeter length that goes from end to end of the curved arms that compose it, where this perimeter length coincides with the sum of the length of the front horizontal channel of the clamp plus the length of the two horizontal recesses of the intermediate portion of the locking button, so that once said locking button has been laterally coupled to the clamp, it is in said horizontal front channel and horizontal recesses that are aligned where the mentioned perimeter closure ring is snap-fitted which has flexible characteristics due to its curved arms, so that when pushed horizontally at the height of the recesses the arms open slightly and fit around the locking button, preventing the latter from moving laterally outward and disengaging from the clamp thus achieving a secure closure of the device.

As it is possible to notice, the central axial hole of the clamp is axially aligned with the axial central hole of the upper portion of the locking button and it is through them that the installed catheter passes; in turn, the semicircular outer notch of the perimeter closure ring is vertically aligned with the radial straight recess of the upper portion of the locking button, so that once the device has been fixed around the distal portion of the catheter that protrudes from the skin of the patient outwards, it causes that this catheter cannot move axially ensuring its closeness to the skin of the abdomen and as it is possible to verify, the clamp can move axially around the protruding catheter as desired, such that allows a universal fixation at the desired point even being able to be adjusted in a millimeter way until the doctor or the person who is mounting it considers that it is properly positioned and there is no axial movement of the catheter beyond what is necessary.

The mounting device being thus fixed around the catheter, the entire portion that protrudes upward from the locking button can be folded and guided by the radial groove that originates from the central perforation and extends to the outside where it coincides vertically with the notch of the perimeter closure ring, so that the catheter passes through said upper groove and can be folded downward guided by said notch, and then bent and inserted into the hook arranged in one of the lower flaps.

The materiality of the device will depend on the production method. If the product is manufactured by 3D printing, it can be manufactured in normal or medical grade ABS filament since the material requirements are less strict when being in contact externally. In case of being manufactured by injection, it can be manufactured with medical grade polypropylene, medical grade polycarbonate or medical grade vinyl polychloride.

### DESCRIPTION OF THE FIGURES

A detailed description of the invention will be carried out in conjunction with the figures that form an integral part of this presentation, where:

FIG. 1 shows an isometric view of the mounting device applied to a Foley type catheter with two upper branches.

FIG. 2 shows an isometric view of the mounting device in an assembled state.

FIG. 3 shows an exploded isometric view of the assembly device in a disassembled state.

FIG. 4 shows an isometric view of the clamp that is part of the device in a disengaged state.

FIG. 5 shows an isometric view of the locking button that is part of the device in a disengaged state.

FIG. 6 shows an isometric view of the closure ring that forms part of the device in a disengaged state.

FIG. 7 shows an isometric view of the clamp that is part of the device in a coupling state.

FIG. 8 shows a top plan view of the clamp that is part of the device in a coupling state.

FIG. 9 shows a front elevation view of the clamp that is part of the device in a coupling state.

FIG. 10 shows a front isometric view of the locking button that is part of the device.

FIG. 11 shows a rear isometric view of the locking button that is part of the device.

FIG. 12 shows a front elevation view of the locking button that is part of the device.

FIG. 13 shows a rear elevation view of the locking button that is part of the device.

FIG. 14 shows a right side elevation view of the locking button that is part of the device.

FIG. 15 shows a front isometric view of the closure ring that is part of the device.

FIG. 16 shows a top plan view of the closure ring that is part of the device.

FIG. 17 shows an isometric view of the locking button and the closure ring that are part of the device in a state prior to coupling.

FIG. 18 shows an isometric view of the locking button and the closure ring that are part of the device in a coupling state.

FIG. 19 shows an exploded isometric view of the device with the parts in a disassembled state prior to assembly.

FIG. 20 shows an exploded isometric view of the device with the parts in a disassembled state, in the clamp engagement step.

FIG. 21 shows an exploded isometric view of the device with the parts in a disassembled state, in the step of engagement of the locking button on the clamp.

FIG. 22 shows an isometric view of the device with the parts in an assembled state, with the locking button on the clamp and the closure ring attached to the locking button.

FIG. 23 shows an isometric view of the device with the parts in an assembled state installed around a catheter that has its retention balloon inflated.

### DETAILED DESCRIPTION OF THE PREFERRED EMBODIMENT

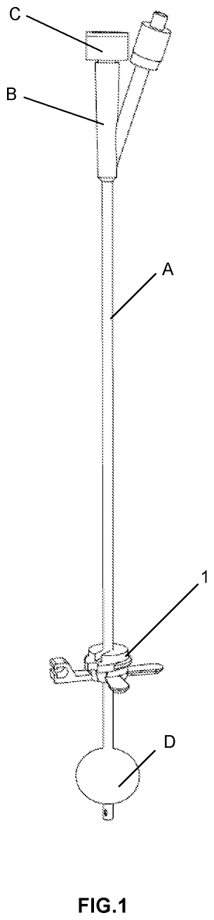

As best illustrated in FIG. 1, the present invention refers to a mounting device (1) for adapting a Foley urinary catheter (A) as a percutaneous endoscopic gastrostomy (PEG) catheter for enteral feeding, which in this case, said catheter has two upper branches (B), where an anti-reflux button (C) is connected to one of them and at its distal end it comprises an internal retaining balloon (D).

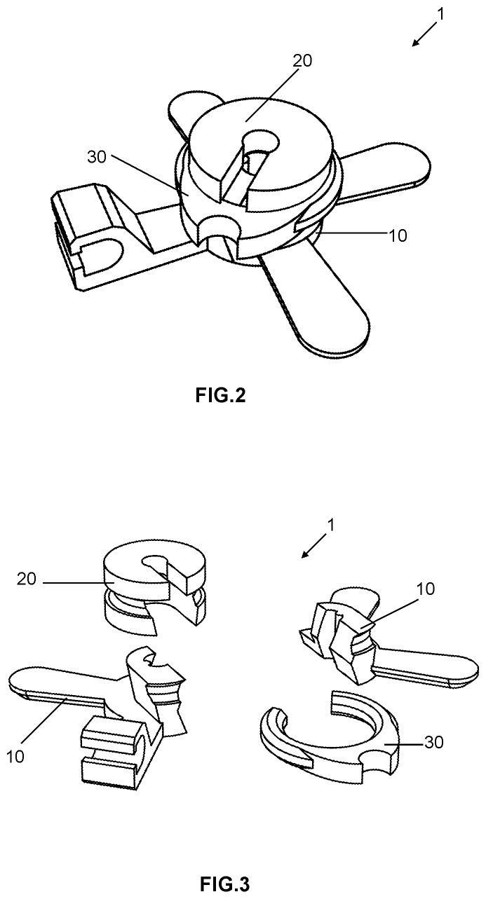

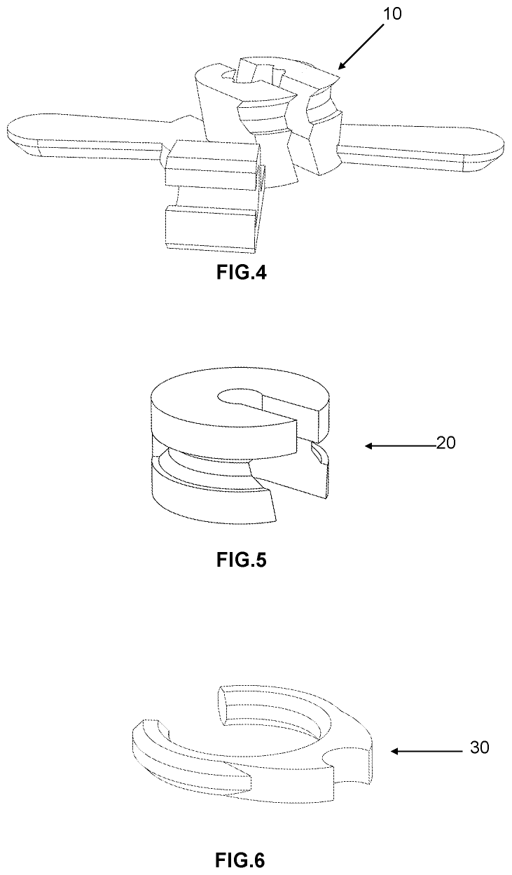

With reference to what is shown in FIG. 2 to FIG. 6, the device (1) comprises a radial clamp-type retention means (10) of the catheter to prevent it from migrating into the patient's stomach cavity, where said clamp (10) is secured in position by a locking button (20) for lateral coupling to the catheter and by a perimeter closure ring (30) that is inserted by pressure outside the locking button (20).

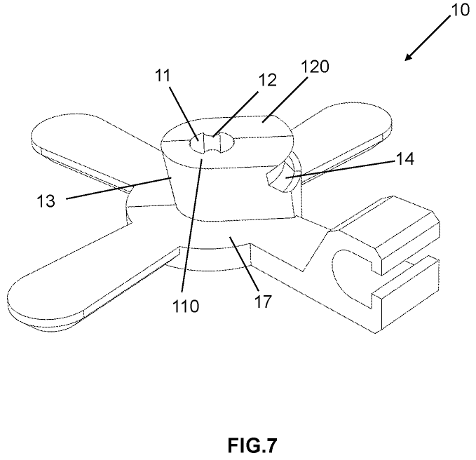

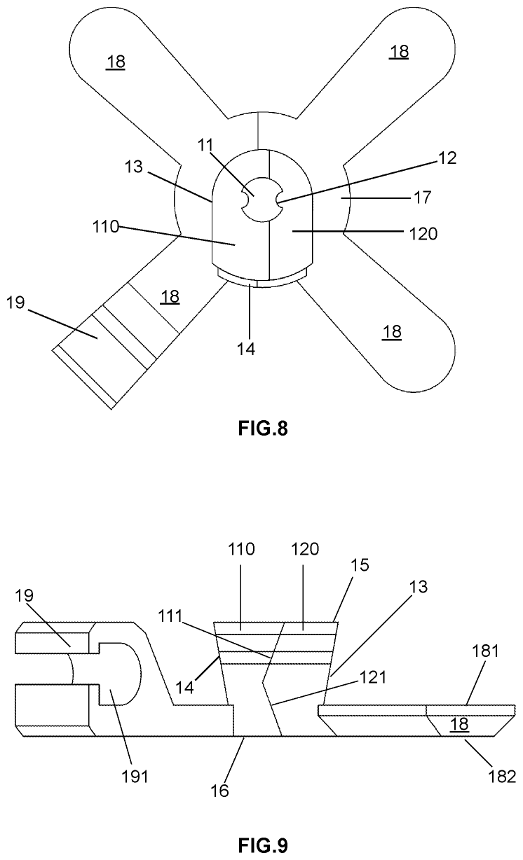

As best illustrated in FIG. 7 and FIG. 8, said clamp (10) has a central axial hole (11) having internal radial compression grooves (12) that stabilize the catheter to prevent its axial displacement and migration towards the interior of the abdominal cavity, where the interior grooves (12) have a semicircular shape section.

With reference to FIG. 8 in combination with FIG. 9, it is seen that the clamp (10) comprises means for retaining the locking button (20) and means for receiving the perimeter closure ring (30), where the means for retention of the locking button (20) comprise an outer wall (13) of the clamp (10), where said wall has a rounded contour and with a convergent negative inclination from top to bottom (see FIG. 9) and the means for reception of the ring (30) comprises a front horizontal channel (14) with a curved path arranged in the front upper edge of said clamp (10), which can also be seen in FIG. 7.

As best seen in FIG. 9, the clamp (10) comprises an upper face (15) and a lower face (16) from whose edges a perimeter spigot (17) projects; while the clamp is divided into a major base piece (110) laterally attachable to a minor base piece (120), where the major base piece (110) has an inner wall (111) facing an inner wall (121) of the minor base piece (120); thus, the inner wall (111) of the major base piece (110) is folded inward at a concave angle, which is complementary to the inner wall (121) of the minor base piece (120), which is folded outward at a convex angle.

Again referring to FIG. 8 in combination with FIG. 9, the major base piece (110) and the minor base piece (120) comprise outer radial flaps (18) projecting from the perimeter spigot (17) that comprise an upper face (181) and a lower opposite face (182) that is in contact with the external abdominal wall of the patient and where one of the flaps (18) comprises at a distal end a hook (19) with a through hole (191) for holding the catheter in the folded state.

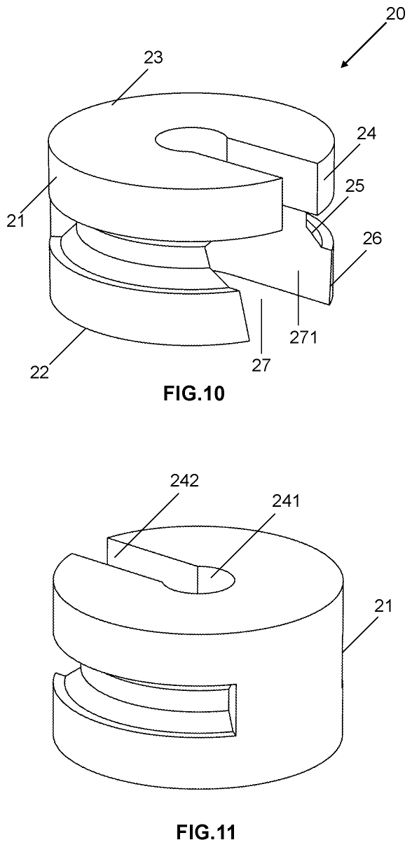

As seen in FIG. 10 and FIG. 11, said locking button (20) comprises a substantially cylindrical body with an outer mantle (21), a lower face (22) and an upper face (23), in which body an upper portion (24) can be distinguished, an intermediate portion (25) and a lower portion (26), where the upper portion (24) comprises an axial central hole (241) from which a radial straight recess (242) extends to the outer mantle (21).

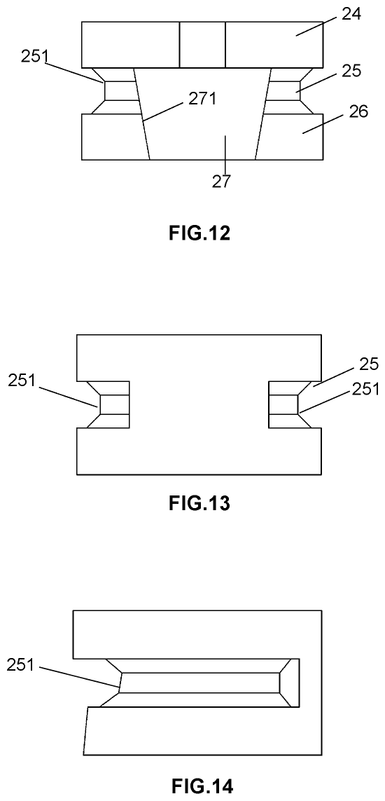

As best illustrated in FIG. 12, the intermediate portion (25) and the lower portion (26) comprise a radially inwardly extending recess (27), which has an interior wall (271) with divergent positive inclination from bottom to top; meanwhile, as seen in FIG. 13 and FIG. 14, the intermediate portion (25) comprises two horizontal recesses (251) opposed to one another, where each of them extends perimeter for approximately a quarter of a circumference from the entrance of the radial recess (27) backward.

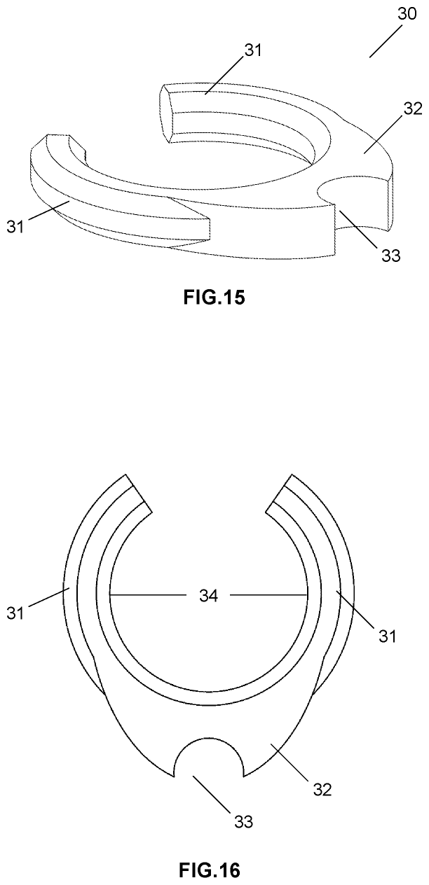

As can be seen in FIG. 15 and FIG. 16, the aforementioned perimeter closure ring (30) has the shape of an open hoop with an internal diameter (34), in which ring (30) two curved arms (31) are distinguished and a front projection (32) with a semicircular outer notch (33) through which the catheter passes.

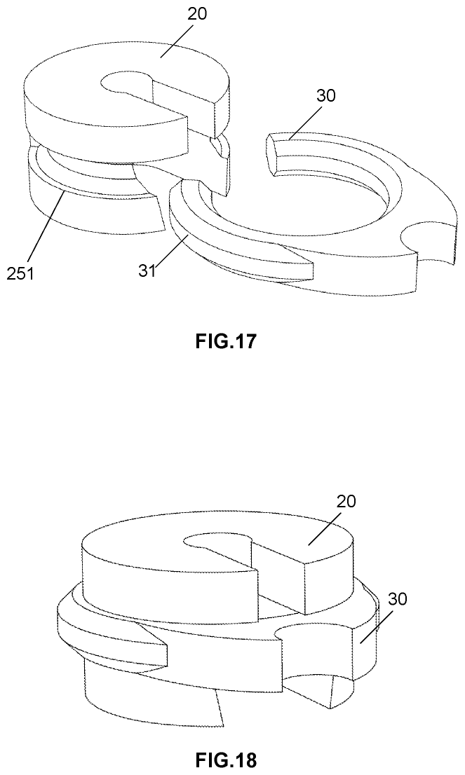

As shown in FIG. 17 and FIG. 18, in the process of coupling the ring (30) around the locking button (20), the curved arms (31) of the ring (30) are arranged and engaged in the horizontal recesses (251) of the locking button (20).

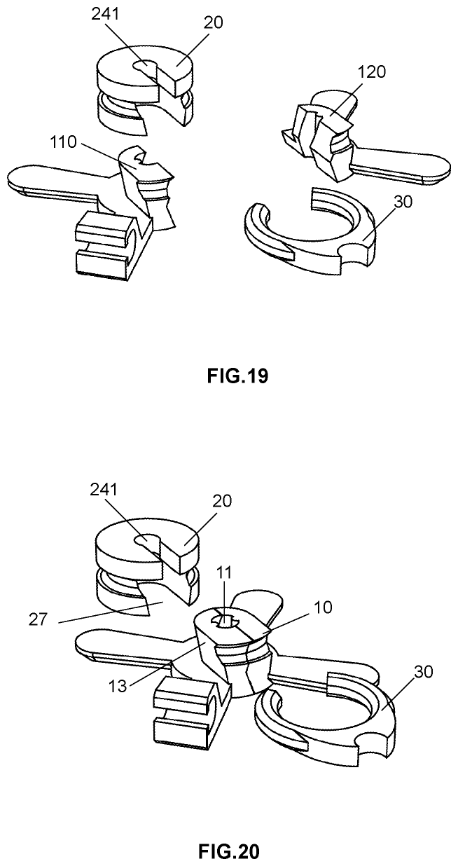

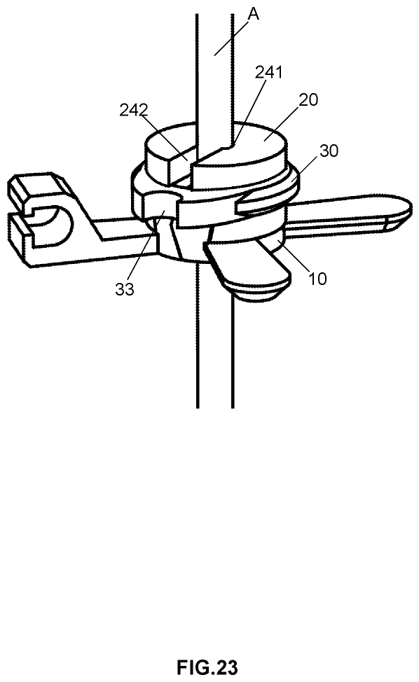

And as best seen from FIG. 19 to FIG. 23, the two base pieces (110) and (120) of the clamp (10) are laterally coupled to each other embracing the catheter (A), so that the internal diameter of the central axial hole (11) of the clamp (10), coincides with the size of the catheter (A) which it retains; in turn, the central axial hole (11) of the clamp (10) is axially aligned with the axial central hole (241) of the upper portion (24) of the locking button (20). The shape, size and inclination of the outer wall (13) of the clamp (10), coincides with the shape, size and inclination of the radial recess (27) of the locking button (20), allowing it to engage laterally in the clamp (10) and is retained therein by effect of the inclined walls preventing its axial displacement.

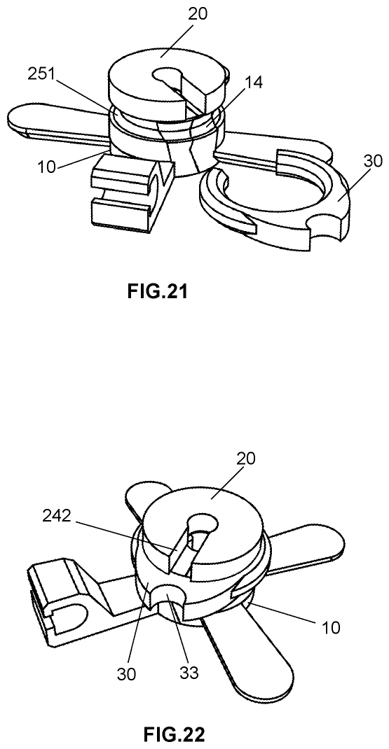

As best seen in FIG. 21, the front horizontal channel (14) arranged in the front upper edge of the clamp (10) is aligned in height with the two horizontal recesses (251) of the locking button (20) and the total length of said aligned channels and recesses corresponds to the total perimeter length of the perimeter closure ring (30). The semicircular outer notch (33) of the perimeter closure ring (30) is vertically aligned with the radial straight recess (242) of the locking button (20).

1.-25. (canceled)

26. A mounting device for adaptation of a catheter (A) for enteral feeding, wherein the mounting device comprises a radial clamp-type retention means of the catheter to prevent it from migrate into the stomach cavity of the patient, where said clamp is secured in position by a locking button, wherein the locking button is for lateral attachment to the catheter, wherein said clamp comprises external radial flaps projecting from a perimeter spigot of the clamp and wherein at least one of the flaps comprises at a distal end a hook with a through hole to hold the catheter in the folded state.

27. The mounting device according to claim 26, wherein the clamp is also secured by a perimeter closure ring that is inserted by pressure outside the locking button.

28. The mounting device according to claim 26, wherein said clamp has a central axial hole having internal radial compression grooves that stabilize the catheter to prevent its axial displacement and migration into the abdominal cavity.

29. The mounting device according to claim 28, wherein the inner grooves have a semicircular section, a triangular shaped section, or a trapezoidal section.

30. The mounting device according to claim 27, wherein said clamp comprises means for retaining the locking button and means for receiving the perimeter closure ring, wherein the means for retaining the locking button can comprise an outer wall of the clamp, where said wall has a rounded contour and with a convergent negative slope from top to bottom, and wherein the means for receiving the ring can comprise a front horizontal channel with a curved path arranged on the upper front edge of said clamp.

31. The mounting device according to claim 26, wherein said clamp comprises an upper face and a lower face from whose edges the perimeter spigot projects, wherein said clamp can be divided into a major base piece laterally attachable to a minor base piece, where the major base piece has an inner wall facing an inner wall of the minor base piece.

32. The mounting device according to claim 31, wherein the inner wall of the major base piece is folded inward at a concave angle, which is complementary to the inner wall of the minor base piece, which is folded outward at a convex angle.

33. The mounting device according to claim 31, wherein the flaps are comprised of the major base piece and the minor base piece, so that the flaps may comprise an upper face and a lower opposite face that is in contact with the external abdominal wall of the patient.

34. The mounting device according to claim 26, wherein the locking button comprises a cylindrical body with an outer mantle, a lower face and an upper face, in which body distinguishes an upper portion, an intermediate portion and a lower portion.

35. The mounting device according to claim 34, wherein the upper portion comprises an axial central hole from which a radial straight recess extends to the outer mantle.

36. The mounting device according to claim 34, wherein the intermediate portion and the lower portion comprise a radial recess of the inner wall with divergent positive inclination from bottom to top.

37. The mounting device according to claim 36, wherein the intermediate portion comprises two horizontal recesses opposed to one another, where each one of them extends perimeter for approximately a quarter of a circumference from the entrance of the radial recess backward.

38. The mounting device according to claim 27, wherein the perimeter closure ring has the shape of an open hoop with an inner diameter, in which ring there are two curved arms and a front projection with a semicircular outer notch through which the catheter passes.

39. The mounting device according to claim 28, wherein the internal diameter of the central axial hole of the clamp, coincides with the size of the catheter it retains.

40. The mounting device according to claim 39, wherein said inner diameter of the central axial hole varies between 5 mm. and 6 mm., being preferably 4 mm.

41. The mounting device according to claim 30, wherein the shape, size and inclination of the outer wall of the clamp, coincides with the shape, size and inclination of a radial recess of the locking button, allowing the locking button to engage laterally in the clamp.

42. The mounting device according to claim 30, wherein the front horizontal channel arranged on the front upper edge of the clamp is aligned in height with two horizontal recesses of the intermediate portion of the locking button and where the total length of said aligned channels and recesses corresponds to the total perimeter length of the perimeter closure ring.

43. The mounting device according to claim 28, wherein the central axial hole of the clamp is axially aligned with an axial central hole of an upper portion of the locking button.

44. The mounting device according to claim 43, wherein the semicircular outer notch of the perimeter closure ring is vertically aligned with a straight radial recess of an upper portion of the locking button.
